(19) United States
(12) Patent Application Publication
(10) Pub. No.: US 2012/0225099 A1
(43) Pub. Date: Sep. 6, 2012

(54) COMPOSITIONS FOR AND METHODS OF ACTIVATING GUANYLYL CYCLASE C

(75) Inventors: Scott A. Waldman, Ardmore, PA (US); Giovanni Pitari, Philadelphia, PA (US); Tong Li, Pennington, NJ (US)

(73) Assignee: Thomas Jefferson University, Philadelphia, PA (US)

(21) Appl. No.: 13/378,658

(22) PCT Filed: Feb. 24, 2010
(86) PCT No.: PCT/US10/25257

§ 371 Date: May 18, 2012

Related U.S. Application Data
(60) Provisional application No. 61/187,128, filed on Jun. 15, 2009.

(51) Int. Cl. A61K 38/02; A61K 9/00; A61P 1/00; A61K 38/16; A61P 35/00; C12N 1/21; A61K 38/10

(52) U.S. Cl. 424/400; 435/252.3; 514/1.1; 514/19.3; 514/21.4; 514/21.5; 514/21.3

(57) ABSTRACT

Isolated non-pathogenic bacterium which comprises a nucleic acid molecule that encodes guanylyl cyclase C agonist operably linked to regulatory sequences operable in bacterium and isolated bacterium which comprises a nucleic acid molecule that encodes guanylyl cyclase C agonist operably linked to inducible regulatory sequences operable in bacterium are disclosed. Isolated culture of such bacteria and uses thereof in the prevention and treatment of colonic polyps and/or colorectal cancer as well as methods of activating guanylyl cyclase C in humans are disclosed.

### BACKGROUND OF THE INVENTION

Guanylyl cyclase C (GCC) is a cell receptor reported to be present in the brush border cells of the bowel in humans. The coding sequence of human gene is disclosed in Genbank Accession Number NM_{—}004963, which is incorporated herein by reference.

Guanylin and uroguanylin are native ligands of GCC. The ligands are small peptides which bind to GCC and have agonist activity.

The heat stable enterotoxin produced by E. coli, referred to as ST, binds to GCC as well. ST binding to GCC is at a much higher affinity and the result is diarrhea. E. coli that produces ST is responsible for what is known as travelers diarrhea. Among infants, elderly and other vulnerable individuals, the diarrhea caused by ST can be lethal.

The expression of GCC by colorectal cancer cells make it a useful market to detect and target metastatic colorectal cancer. In addition, GCC agonists have been disclosed for use in the treatment of primary colorectal cancer, and autoimmune diseases such as inflammatory bowel disease. Such GCC agonists includes anti-GCC antibodies, ST peptides and related toxins, guanylin, uroguanylin and modified forms of such peptides. The delivery of GCC agonists to the bowel is useful in the prevention of formation of polyps, in the prevention of development of polyps in colorectal cancer, in the treatment of primary colorectal cancer, in the treatment of autoimmune diseases involving the colon such as inflammatory bowel disease.

There remains a need for more effective compositions and methods for delivering GCC agonists to the bowel. There remains a need for more effective compositions and methods useful in the prevention of formation of polyps, in the prevention of development of polyps in colorectal cancer, in the treatment of primary colorectal cancer, in the treatment of autoimmune diseases involving the colorectal track such as inflammatory bowel disease.

### SUMMARY OF THE INVENTION

The present invention relates to isolated non-pathogenic bacterium which comprises a nucleic acid molecule that encodes guanylyl cyclase C agonist operably linked to regulatory sequences operable in the bacterium and to isolated cultures of such bacteria. The bacterium is of a species that can live in a human colon as part of a human's gut flora.

The present invention further relates to isolated non-pathogenic bacterium which comprises a nucleic acid molecule that encodes guanylyl cyclase C agonist operably linked to regulatory sequences operable in the bacterium including an inducible promoter and to isolated cultures of such bacteria. The bacterium is of a species that can live in a human colon as part of a human's gut flora.

The present invention additionally relates to isolated bacterium comprising a nucleic acid molecule encoding a GCC agonist operably linked to inducible regulatory elements. Regulatory elements are inducible and to isolated cultures of such bacteria.

The present invention also relates to compositions comprising a guanylyl cyclase C agonist formulated for large intestine specific release when administered orally to an individual.

The present invention relates to method of preventing colonic polyps in an individual who has been identified as being at high risk for colonic polyps. The methods comprise the step of administering to the individual isolated culture of non-pathogenic bacteria that express GCC agonists and/or bacteria that express GCC agonists under the control of inducible regulatory elements, or a composition comprising a guanylyl cyclase C agonist formulated for large intestine specific release when administered orally to an individual.

The present invention relates to method of treating an individual who has been diagnosed with colonic polyps. The methods comprise the step of administering to the individual isolated culture of non-pathogenic bacteria that express GCC agonists and/or bacteria that express GCC agonists under the control of inducible regulatory elements, or a composition comprising a guanylyl cyclase C agonist formulated for large intestine specific release when administered orally to an individual.

The present invention relates to method of preventing colorectal cancer in an individual who has been identified as being at high risk for colorectal cancer. The methods comprise the step of administering to the individual isolated culture of non-pathogenic bacteria that express GCC agonists and/or bacteria that express GCC agonists under the control of inducible regulatory elements, or a composition comprising a guanylyl cyclase C agonist formulated for large intestine specific release when administered orally to an individual.

The present invention further relates to method of treating an individual who has been diagnosed with colorectal cancer. The methods comprise the step of administering to the individual isolated culture of non-pathogenic bacteria that express GCC agonists and/or bacteria that express GCC agonists under the control of inducible regulatory elements, or a composition comprising a guanylyl cyclase C agonist formulated for large intestine specific release when administered orally to an individual.

The present invention also relates to methods of inducing guanylyl cyclase C activity in the cells of an individual's colon. The methods comprise the step of administering to the individual isolated culture of non-pathogenic bacteria that express GCC agonists and/or bacteria that express GCC agonists under the control of inducible regulatory elements, or a composition comprising a guanylyl cyclase C agonist formulated for large intestine specific release when administered orally to an individual.

### DESCRIPTION OF PREFERRED EMBODIMENTS

As used herein, the terms “colorectal” and “colorectal track” are meant to refer to colon, bowel, large intestine, rectum sphincter and anus. That is, “colorectal” and “colorectal track” refer to tissues and organs of the digestive and excretory systems below/“downstream” from the small intestine.

As used herein, non-pathogenic bacterium is meant to refer to a species and strains of bacterium which normally populate a human's gut without pathogenic effects. Expressly excluded from the meaning of non-pathogenic bacterium are those species which are known to be responsible to enterogenic diarrhea such as specific strains of E. coli comprise coding sequences for heat stable enterotoxin, Yersinia, and cholerae.

As used herein the terms guanylyl cyclase C agonist and “GCC agonists” are used interchangeably and refer to molecules which bind to guanylyl cyclase C and thereby induce its activity.

As used herein, operably linked to regulatory sequences operable in said bacterium is meant to refer to the condition in which a coding sequence is linked to regulatory elements sufficient for it be functional in the host bacterium. With respect to operable promoters, a coding sequenced is operably linked to regulatory sequences operable in said bacterium if the coding sequence can be expressed in the bacterium in the presence of the inducer.

As used herein, formulated for large intestine specific release when administered orally to an individual is meant to refer to the condition by which the guanylyl cyclase C agonist is generally not released in the stomach or small intestine of the individual but the guanylyl cyclase C agonist becomes available in the large intestine.

Current methods for delivering GCC agonists to the colorectal track involved the oral delivery of such GCC agonists. ST peptides and other GCC agonist peptides, for example, are stable and can survive the stomach acid and pass through the small intestine to the colorectal track. While they can reach the colorectal track intact and able to positively interact with cells that express GCC, their presence in the small intestine can have serious side effects. Moreover, regular dosing is required to maintain an effective level of GCC agonist in the colorectal track, such dosing further exacerbating the side effects caused by the GCC agonists passing through the small intestine.

One solution to this problem is to provide a composition that comprises the GCC agonist in a form that is inactive until it reaches the colorectal track. Such embodiments include coating or otherwise encapsulating the GCC agonist such that the GCC agonist is not released until it passes through the small intestine.

Another solution to this problem is to provide a bacteria culture comprising innocuous bacteria which include coding sequences that encode a GCC agonist in an expressible form. In such embodiments, the bacteria may colonize the colorectal track and, express the GCC agonist, thereby delivering it to the colorectal track. In some embodiments, the coding sequences that encode the GCC agonist may be linked to an inducible regulatory element, such as an inducible promoter, so that the bacteria may colonize the colorectal track and, upon delivery of the inducing agent, express the GCC agonist, thereby delivering it to the colorectal track.

These solutions not only eliminate unwanted side effects associated with GCC agonist activity in the small intestine, they also allow more controlled and effective delivery of the GCC agonist to the colorectal track. By elimination of the side effects associated with GCC agonist activity in the small intestine, more GCC agonist can be delivered to colorectal track, providing an improved beneficial effect relative to standard oral delivery. Bypassing the stomach and small intestine allows for the amount of GCC agonist delivered to the colorectal track to be tolerated at higher doses, and in the case of using bacteria as a delivery vector, the ability to deliver GCC agonists over long periods of time.

GCC agonists are known. Two native GCC agonists, guanylin and uroguanylin, have been identified (see U.S. Pat. Nos. 5,969,097 and 5,489,670, which are each incorporated herein by reference. In addition, several small peptides, which are produced by enteric pathogens, are toxigenic agents which cause diarrhea (see U.S. Pat. No. 5,518,888, which is incorporated herein by reference). The most common pathogen derived GCC agonist is the heat stable entertoxin produced by strains of pathogenic E. coli. Native heat stable enterotoxin produced by pathogenic E coli is also referred to as ST. A variety of other pathogenic organisms including Yersinia and Enterobacter, also make enterotoxins which can bind to guanylyl cyclase C in an agonistic manner. In nature, the toxins are generally encoded on a plasmid which can “jump” between different species. Several different toxins have been reported to occur in different species. These toxins all possess significant sequence homology, they all bind to ST receptors and they all activate guanylate cyclase, producing diarrhea.

ST has been both cloned and synthesized by chemical techniques. The cloned or synthetic molecules exhibit binding characteristics which are similar to native ST. Native ST isolated from E. coli is 18 or 19 amino acids in length. The smallest “fragment” of ST which retains activity is the 13 amino acid core peptide extending toward the carboxy terminal from cysteine 6 to cysteine 18 (of the 19 amino acid form). Analogues of ST have been generated by cloning and by chemical techniques. Small peptide fragments of the native ST structure which include the structural determinant that confers binding activity may be constructed. Once a structure is identified which binds to ST receptors, non-peptide analogues mimicking that structure in space are designed.

SEQ ID NO:1 discloses a nucleotide sequence which encodes 19 amino acid ST, designated ST Ia, reported by So and McCarthy (1980) Proc. Natl. Acad. Sci. USA 77:4011, which is incorporated herein by reference.

The amino acid sequence of ST Ia is disclosed in SEQ ID NO:2.

SEQ ID NO:3 discloses the amino acid sequence of an 18 amino acid peptide which exhibits ST activity, designated ST I*, reported by Chan and Giannella (1981) J. Biol. Chem. 256:7744, which is incorporated herein by reference.

SEQ ID NO:4 discloses a nucleotide sequence which encodes 19 amino acid ST, designated ST Ib, reported by Mosely et al. (1983) Infect. Immun. 39:1167, which is incorporated herein by reference.

The amino acid sequence of ST Ib is disclosed in SEQ ID NO:5.

A 15 amino acid peptide called guanylin which has about 50% sequence homology to ST has been identified in mammalian intestine (Currie, M. G. et al. (1992) Proc. Natl. Acad. Sci. USA 89:947-951, which is incorporated herein by reference). Guanylin binds to ST receptors and activates guanylate cyclase at a level of about 10- to 100-fold less than native ST. Guanylin may not exist as a 15 amino acid peptide in the intestine but rather as part of a larger protein in that organ. The amino acid sequence of guanylin from rodent is disclosed as SEQ ID NO:6.

SEQ ID NO:7 is an 18 amino acid fragment of SEQ ID NO:2. SEQ ID NO:8 is a 17 amino acid fragment of SEQ ID NO:2. SEQ ID NO:9 is a 16 amino acid fragment of SEQ ID NO:2. SEQ ID NO:10 is a 15 amino acid fragment of SEQ ID NO:2. SEQ ID NO:11 is a 14 amino acid fragment of SEQ ID NO:2. SEQ ID NO:12 is a 13 amino acid fragment of SEQ ID NO:2. SEQ ID NO:13 is an 18 amino acid fragment of SEQ ID NO:2. SEQ ID NO:14 is a 17 amino acid fragment of SEQ ID NO:2. SEQ ID NO:15 is a 16 amino acid fragment of SEQ ID NO:2. SEQ ID NO:16 is a 15 amino acid fragment of SEQ ID NO:2. SEQ ID NO:17 is a 14 amino acid fragment of SEQ ID NO:2.

SEQ ID NO:18 is a 17 amino acid fragment of SEQ ID NO:3. SEQ ID NO:19 is a 16 amino acid fragment of SEQ ID NO:3. SEQ ID NO:20 is a 15 amino acid fragment of SEQ ID NO:3. SEQ ID NO:21 is a 14 amino acid fragment of SEQ ID NO:3. SEQ ID NO:22 is a 13 amino acid fragment of SEQ ID NO:3. SEQ ID NO:23 is a 17 amino acid fragment of SEQ ID NO:3. SEQ ID NO:24 is a 16 amino acid fragment of SEQ ID NO:3. SEQ ID NO:25 is a 15 amino acid fragment of SEQ ID NO:3. SEQ ID NO:26 is a 14 amino acid fragment of SEQ ID NO:3.

SEQ ID NO:27 is an 18 amino acid fragment of SEQ ID NO:5. SEQ ID NO:28 is a 17 amino acid fragment of SEQ ID NO:5. SEQ ID NO:29 is a 16 amino acid fragment of SEQ ID NO:5. SEQ ID NO:30 is a 15 amino acid fragment of SEQ ID NO:5. SEQ ID NO:31 is a 14 amino acid fragment of SEQ ID NO:5. SEQ ID NO:32 is a 13 amino acid fragment of SEQ ID NO:5. SEQ ID NO:33 is an 18 amino acid fragment of SEQ ID NO:5. SEQ ID NO:34 is a 17 amino acid fragment of SEQ ID NO:5. SEQ ID NO:35 is a 16 amino acid fragment of SEQ ID NO:5. SEQ ID NO:36 is a 15 amino acid fragment of SEQ ID NO:5. SEQ ID NO:37 is a 14 amino acid fragment of SEQ ID NO:5.

SEQ ID NO:27, SEQ ID NO:31, SEQ ID NO:36 AND SEQ ID NO:37 are disclosed in Yoshimura, S., et al. (1985) FEBS Lett. 181:138, which is incorporated herein by reference.

SEQ ID NO:38, SEQ ID NO:39 and SEQ ID NO:40, which are derivatives of SEQ ID NO:3, are disclosed in Waldman, S. A. and O'Hanley, P. (1989) Infect. Immun. 57:2420, which is incorporated herein by reference.

SEQ ID NO:41, SEQ ID NO:42, SEQ ID NO:43, SEQ ID NO:44 and SEQ ID NO:45, which are a derivatives of SEQ ID NO:3, are disclosed in Yoshimura, S., et al. (1985) FEBS Lett. 181:138, which is incorporated herein by reference.

SEQ ID NO:46 is a 25 amino acid peptide derived from Y. enterocolitica which binds to the ST receptor.

SEQ ID NO:47 is a 16 amino acid peptide derived from V. cholerae which binds to the ST receptor. SEQ ID NO:47 is reported in Shimonishi, Y., et al. FEBS Lett. 215:165, which is incorporated herein by reference.

SEQ ID NO:48 is an 18 amino acid peptide derived from Y. enterocolitica which binds to the ST receptor. SEQ ID NO:48 is reported in Okamoto, K., et al. Infec. Immun. 55:2121, which is incorporated herein by reference.

SEQ ID NO:49, is a derivative of SEQ ID NO:5. SEQ ID NO:50, SEQ ID NO:51, SEQ ID NO:52 and SEQ ID NO:53 are derivatives. SEQ ID NO:54 is the amino acid sequence of guanylin from human.

A 15 amino acid peptide called uroguanylin has been identified in mammalian intestine from opossum (Hamra, S. K. et al. (1993) Proc. Natl. Acad. Sci. USA 90:10464-10468, which is incorporated herein by reference; see also Forte L. and M. Curry 1995 FASEB 9:643-650; which is incorporated herein by reference). SEQ ID NO:55 is the amino acid sequence of uroguanylin from opossum.

A 16 amino acid peptide called uroguanylin has been identified in mammalian intestine from human (Kita, T. et al. (1994) Amer. J. Physiol. 266:F342-348, which is incorporated herein by reference; see also Forte L. and M. Curry 1995 FASEEB 9:643-650; which is incorporated herein by reference). SEQ ID NO:56 is the amino acid sequence of uroguanylin from human.

In some preferred embodiments, non-pathogenic bacteria are engineered to express a GCC agonsiot that comprise amino acid sequences selected from the group consisting of SEQ ID NO:2, SEQ ID NO:3, SEQ ID NOS:5-56 and fragments and derivatives thereof.

SEQ ID NO:57 is the amino acid sequence of proguanylin, a guanylin precursor which is processed into active guanylin.

SEQ ID NO:58 is the amino acid sequence of prouroguanylin, a uroguanylin precursor which is processed into active uroguanylin.

Although proguanylin and prouroguanylin are precursors for mature guanylin and mature uroguanylin respectively, they may be used as GCC agonists as described herein provide they are delivered such that they can be processed into the mature peptides.

U.S. Pat. Nos. 5,140,102, 7,041,786 and 7,304,036, and U.S. Published Applications US 2004/0258687, US 2005/0287067, 20070010450, 20040266989, 20060281682, 20060258593, 20060094658, 20080025966, 20030073628, 20040121961 and 20040152868, which are each incorporated herein by reference, also refer to compounds which may bind to and activate guanylyl cyclase C.

Most enteric coatings are intended to protect contents from stomach acid. Accordingly, they are designed to release active agent upon passing through the stomach. The coatings and encapsulations used herein are provided to release the GCC agonist upon passing the small intestine. This can be accomplished in several ways.

According to some embodiments, the GCC agonists are coated or encapsulated with a sufficient amount of coating material that the time required for the coating material to dissolve and release the GCC agonists corresponds with the time required for the coated or encapsulated composition to travel from the mouth to the colorectal track.

According to some embodiments, the GCC agonists are coated or encapsulated with coating material that does not fully dissolve and release the GCC agonists until it comes in contact with conditions present in the colorectal track. Such conditions may include the presence of enzymes in the colorectal track, pH, tonicity, or other conditions that vary relative to the small intestine.

According to some embodiments, the GCC agonists are coated or encapsulated with coating material that is designed to dissolve in stages as it passes from stomach to small intestine to large intestine. GCC agonists are released upon dissolution of the final stage which occurs in the colorectal track.

According to some embodiments, the GCC agonists are complexed with another molecular entity such that they are inactive until the GCC agonists cease to be complexed with molecular entity and are present in active form. In such embodiments, the GCC agonists are administered as “prodrugs” which become processed into active GCC agonists in the colorectal track.

Examples of technologies which may be used to formulate GCC agonists or inducers for large intestine specific release when administered include, but are not limited to: U.S. Pat. No. 5,108,758 issued to Allwood, et al. on Apr. 28, 1992 which discloses delayed release formulations; U.S. Pat. No. 5,217,720 issued to Sekigawa, et al. on Jun. 8, 1993 which discloses coated solid medicament form having releasability in large intestine; U.S. Pat. No. 5,541,171 issued to Rhodes, et al. on Jul. 30, 1996 which discloses orally administrable pharmaceutical compositions; U.S. Pat. No. 5,688,776 issued to Bauer, et al. on Nov. 18, 1997 which discloses crosslinked polysaccharides, process for their preparation and their use; U.S. Pat. No. 5,846,525 issued to Maniar, et al. on Dec. 8, 1998 which discloses protected biopolymers for oral administration and methods of using same; U.S. Pat. No. 5,863,910 to Bolonick, et al. on Jan. 26, 1999 which discloses treatment of chronic inflammatory disorders of the gastrointestinal tract; U.S. Pat. No. 6,849,271 to Vaghefi, et al. on Feb. 1, 2005 which discloses microcapsule matrix microspheres, absorption-enhancing pharmaceutical compositions and methods; U.S. Pat. No. 6,972,132 to Kudo, et al. on Dec. 6, 2005 which discloses a system for release in lower digestive tract; U.S. Pat. No. 7,138,143 to Mukai, et al. Nov. 21, 2006 which discloses coated preparation soluble in the lower digestive tract; U.S. Pat. No. 6,309,666; U.S. Pat. No. 6,569,463, U.S. Pat. No. 6,214,378; U.S. Pat. No. 6,248,363; U.S. Pat. No. 6,458,383, U.S. Pat. No. 6,531,152, U.S. Pat. No. 5,576,020, U.S. Pat. No. 5,654,004, U.S. Pat. No. 5,294,448, U.S. Pat. No. 6,309,663, U.S. Pat. No. 5,525,634, U.S. Pat. No. 6,248,362, U.S. Pat. No. 5,843,479, and U.S. Pat. No. 5,614,220, which are each incorporated herein by reference.

According to some aspects of the invention, innocuous bacteria of species that normally populate the colon are provided with genetic information needed to produce a guanylyl cyclase C agonist in the colon, making such guanylyl cyclase C agonist available to produce the effect of activating the guanylyl cyclase C on colon cells, or inhibiting formation of colon polyps, or treating colon polyps, or inhibiting formation of colorectal tumors, or treating colorectal cancer. The existence of a population of bacteria which can produce guanylyl cyclase C agonist provides a continuous administration of the guanylyl cyclase C agonist in the site where it is needed. In some embodiments, the nucleic acid sequences that encode the guanylyl cyclase C agonist are under the control of an inducible promoter. Accordingly, the individual may turn expression on or off depending upon whether or not the inducer is ingested. In some embodiments, the inducer is formulated to be specifically released in the colon, thereby preventing induction of expression by the bacteria that may be populating other sites such as the small intestine. In some embodiments, the bacteria are is sensitive to a particular drug or auxotrophic such that it can be eliminated by administration of the drug or withholding an essential supplement.

The technology for introducing expressible forms of genes into bacteria is well known and the materials needed are widely available.

In some embodiments, bacteria which comprise coding sequences for a GCC agonist may be those of a species which commonly inhabits the intestinal track of an individual. Common gut flora include species from the genera Bacteroides, Clostridium, Fusobacterium, Eubacterium, Ruminococcus, Peptococcus, Peptostreptococcus, Bifidobacteriu, Escherichia and Lactobacillus. In some embodiments, the bacteria is selected from a strain known to be useful as a probiotic. Examples of species of bacteria used as compositions for administration to humans include Bifidobacterium bifidum; Escherichia coli, Lactobacillus acidophilus, Lactobacillus rhamnosus, Lactobacillus casei, and Lactobacillus johnsonii. Other species include Lactobacillus bulgaricus, Streptococcus thermophilus, Bacillus coagulans and Lactobacillus bifidus. Examples of strains of bacteria used as compositions for administration to humans include: B. infantis 35624, (Align); Lactobacillus plantarum 299V; Bifidobacterium animalis DN-173 010; Bifidobacterium animalis DN 173 010 (Activia Danone); Bifidobacterium animalis subsp. lactis BB-12 (Chr.Hansen); Bifidobacterium breve Yakult Bifiene Yakult; Bifidobacterium infantis 35624 Bifidobacterium lactis HNO19 (DR10) Howaru™ Bifido Danisco; Bifidobacterium longum BB536; Escherichia coli Nissle 1917; Lactobacillus acidophilus LA-5 Chr. Hanse;n; Lactobacillus acidophilus NCFM Rhodia Inc.; Lactobacillus casei DN114-001; Lactobacillus casei CRL431 Chr. Hansen; Lactobacillus casei F19 Cultura Arla Foods; Lactobacillus casei Shirota Yakult Yakult; Lactobacillus casei immunitass Actimel Danone; Lactobacillus johnsonnii La1 (=Lactobacillus LC1) Nestlé; Lactobacillus plantarum 299V ProViva Probi IBS; Lactobacillus reuteri ATTC 55730 BioGaia Biologics; Lactobacillus reuteri SD2112; Lactobacillus rhamnosus ATCC 53013 Vifit and others Valio; Lactobacillus rhamnosus LB21 Verum Norrmejerier; Lactobacillus salivarius UCC118; Lactococcus lactis L1A Verum Norrmejerier; Saccharomyces cerevisiae (boulardii) lyo; Streptococcus salivarius ssp thermophilus; Lactobacillus rhamnosus GR-1; Lactobacillus reuteri RC-14; Lactobacillus acidophilus CUL60; Bifidobacterium bifidum CUL 20; Lactobacillus helveticus R0052; and Lactobacillus rhamnosus R0011.

The following U.S. patents, which are each incorporated herein by reference, disclose non-pathogenic bacteria which can be administered to individuals. U.S. Pat. No. 6,200,609; U.S. Pat. No. 6,524,574, U.S. Pat. No. 6,841,149, U.S. Pat. No. 6,878,373, U.S. Pat. No. 7,018,629, U.S. Pat. No. 7,101,565, U.S. Pat. No. 7,122,370, U.S. Pat. No. 7,172,777, U.S. Pat. No. 7,186,545, U.S. Pat. No. 7,192,581, U.S. Pat. No. 7,195,906, U.S. Pat. No. 7,229,818, and U.S. Pat. No. 7,244,424.

Accordingly the aspects of the invention, bacteria would first be provided with genetic material encoding a GCC agonist in a form that would permit expression le of the agonist peptide within the bacteria, either constitutively or upon induction by the presence of an inducer that would turn on an inducible promoter.

Some embodiments comprise inducible regulatory elements such as inducible promoters. Typically, an inducible promoter is one in which an agent, when present, interacts with the promoter such that expression of the coding sequence operably linked to the promoter proceeds. Alternatively, an inducible promoter can include a repressor which is an agent that interacts with the promoter and prevent expression of the coding sequence operably linked to the promoter. Removal of the repressor results in expression of the coding sequence operably linked to the promoter.

The agents that induce an inducible promoter are preferably not naturally present in the organism where expression of the transgene is sought. Accordingly, the transgene is only expressed when the organism is affirmatively exposed to the inducing agent. Thus, in a bacterium that includes a transgene operably linked to an inducible promoter, when the bacterium is living within the gut of an individual, the promoter may be turned on and the transgene expressed when the individual ingests the inducing agent.

The agents that induce an inducible promoter are preferably not toxic. Thus, in a bacterium that includes a transgene operably linked to an inducible promoter, the inducing agent is preferably not toxic to the individual in whose gut the bacterium is living such that when the individual ingests the inducing agent to turn on expression of the transgene the inducing agent dose not have any severe toxic side effects on the individual.

The agents that induce an inducible promoter preferably affect only the expression of the gene of interest. Thus, in a bacterium that includes a transgene operably linked to an inducible promoter, the inducing agent does not have any significant affect on the expression of any other genes in the individual.

The agents that induce an inducible promoter preferably are easy to apply or removal. Thus, in a bacterium that includes a transgene operably linked to an inducible promoter that is living in the gut of an individual, the inducing agent is preferably an agent that can be easily delivered to the gut and that can be removed, either by affirmative neutralization for example or by metabolism/passing such that gene expression can be controlled

The agents that induce an inducible promoter preferably induce a clearly detectable expression pattern of either high or very low gene expression.

In some preferred embodiments, the chemically-regulated promoters are derived from organisms distant in evolution to the organisms where its action is required. Examples of inducible or chemically-regulated promoters include tetracycline-regulated promoters. Tetracycline-responsive promoter systems can function either to activate or repress gene expression system in the presence of tetracycline. Some of the elements of the systems include a tetracycline repressor protein (TetR), a tetracycline operator sequence (tetO) and a tetracycline transactivator fusion protein (tTA), which is the fusion of TetR and a herpes simplex virus protein 16 (VP16) activation sequence. The Tetracycline resistance operon is carried by the Escherichia coli transposon (Tn) 10. This operon has a negative mode of operation. The interaction between a repressor protein encoded by the operon, TetR, and a DNA sequence to which it binds, the tet operator (tetO), represses the activity of a promoter placed near the operator. In the absence of an inducer, TetR binds to tetO and prevents transcription. Transcription can be turned on when an inducer, such as tetracycline, binds to TetR and causes a conformation change that prevents TetR from remaining bound to the operator. When the operator site is not bound, the activity of the promoter is restored. Tetracycline, the antibiotic, has been used to create two beneficial enhancements to inducible promoters. One enhancement is an inducible on or off promoter. The investigators can choose to have the promoter always activated until Tet is added or always inactivated until Tet is added. This is the Tet on/off promoter. The second enhancement is the ability to regulate the strength of the promoter. The more Tet added, the stronger the effect so now you can turn up or down an expression vector the way you turn up or down the volume on a radio.

Examples of inducible or chemically-regulated promoters include Steroid-regulated promoters. Steroid-responsive promoters are provided for the modulation of gene expression include promoters based on the rat glucocorticoid receptor (GR); human estrogen receptor (ER); ecdysone receptors derived from different moth species; and promoters from the steroid/retinoid/thyroid receptor superfamily. The hormone binding domain (HBD) of GR and other steroid receptors can also be used to regulate heterologous proteins in cis, that is, operatively linked to protein-encoding sequences upon which it acts. Thus, the HBD of GR, estrogen receptor (ER) and an insect ecdysone receptor have shown relatively tight control and high inducibility

Examples of inducible or chemically-regulated promoters include metal-regulated promoters. Promoters derived from metallothionein (proteins that bind and sequester metal ions) genes from yeast, mouse and human are examples of promoters in which the presence of metals induces gene expression.

IPTG is a classic example of a compound added to cells to activate a promoter. IPTG can be added to the cells to activate the downstream gene or removed to inactivate the gene.

U.S. Pat. No. 6,180,391, which is incorporated herein by reference, refers to the a copper-inducible promoter.

U.S. Pat. No. 6,943,028, which is incorporated herein by reference, refers to highly efficient controlled expression of exogenous genes in E. coli.

U.S. Pat. No. 6,180,367, which is incorporated herein by reference, refers to a process for bacterial production of polypeptides.

Other examples of inducible promoters suitable for use with bacterial hosts include the beta.-lactamase and lactose promoter systems (Chang et al., Nature, 275: 615 (1978, which is incorporated herein by reference); Goeddel et al., Nature, 281: 544 (1979), which is incorporated herein by reference), the arabinose promoter system, including the araBAD promoter (Guzman et al., J. Bacteriol., 174: 7716-7728 (1992), which is incorporated herein by reference; Guzman et al., J. Bacteriol., 177: 4121-4130 (1995), which is incorporated herein by reference; Siegele and Hu, Proc. Natl. Acad. Sci. USA, 94: 8168-8172 (1997), which is incorporated herein by reference), the rhamnose promoter (Haldimann et al., J. Bacteriol., 180: 1277-1286 (1998), which is incorporated herein by reference), the alkaline phosphatase promoter, a tryptophan (trp) promoter system (Goeddel, Nucleic Acids Res., 8: 4057 (1980), which is incorporated herein by reference), the P.sub.LtetO-1 and P.sub.lac/are-1 promoters (Lutz and Bujard, Nucleic Acids Res., 25: 1203-1210 (1997), which is incorporated herein by reference), and hybrid promoters such as the tac promoter. deBoer et al., Proc. Natl. Acad. Sci. USA, 80: 21-25 (1983), which is incorporated herein by reference. However, other known bacterial inducible promoters and low-basal-expression promoters are suitable.

U.S. Pat. No. 6,083,715, which is incorporated herein by reference, refers to methods for producing heterologous disulfide bond-containing polypeptides in bacterial cells.

U.S. Pat. No. 5,830,720, which is incorporated herein by reference, refers to recombinant DNA and expression vector for the repressible and inducible expression of foreign genes.

U.S. Pat. No. 5,789,199, which is incorporated herein by reference, refers to a process for bacterial production of polypeptides.

U.S. Pat. No. 5,085,588, which is incorporated herein by reference, refers to bacterial promoters inducible by plant extracts.

U.S. Pat. No. 6,242,194, which is incorporated herein by reference, refers to probiotic bacteria host cells that contain a DNA of interest operably associated with a promoter of the invention can be orally administered to a subject . . . .

U.S. Pat. No. 5,364,780, which is incorporated herein by reference, refers to external regulation of gene expression by inducible promoters.

U.S. Pat. No. 5,639,635, which is incorporated herein by reference, refers to a process for bacterial production of polypeptides.

U.S. Pat. No. 5,789,199, which is incorporated herein by reference, refers to a process for bacterial production of polypeptides.

U.S. Pat. No. 5,689,044, which is incorporated herein by reference, refers to chemically inducible promoter of a plant PR-1 gene.

U.S. Pat. No. 5,063,154, which is incorporated herein by reference, refers to a pheromone-inducible yeast promoter.

U.S. Pat. No. 5,658,565, which is incorporated herein by reference, refers to an inducible nitric oxide synthase gene.

U.S. Pat. Nos. 5,589,392, 6,002,069, 5,693,531, 5,480,794, 6,171,816 6,541,224, 6,495,318, 5,498,538, 5,747,281, 6,635,482 and 5,364,780, which are each incorporated herein by reference, each refer to an IPTG-inducible promoters.

U.S. Pat. Nos. 6,420,170, 5,654,168, 5,912,411, 5,891,718, 6,133,027, 5,739,018, 6,136,954, 6,258,595, 6,002,069 and 6,025,543, which are each incorporated herein by reference, each refer to an tetracycline-inducible promoters.

The GCC agonists whether delivered for targeted release or using bacterial expression in vivo, may be provided in combination with other prophylactic or therapeutic compounds. Examples of anti-colorectal drugs which may be used in combination with the compositins and/or bacteria of the present invention include, but are not limited to: Adrucil (fluorouracil, 5-FU); Avastin (bevacizumab); Camptosar (irinotecan); Eloxatin (oxaliplatin); Erbitux; (cetuximab); Vectibix, (panitumumab); Wellcovorin, (leucovorin); and Xeloda (capecitabine); as well as the compounds disclosed in U.S. Pat. No. 5,879,656, U.S. Patent Application 20040258687 and U.S. Patent Application 20050287067, which are each incorporated herein by reference.

A correlation between levels of proguanylin and/or prouroguanylin in the blood and levels of guanylin and/or uroguanylin in the intestine may be used to allow for the determination of proguanylin and/or prouroguanylin levels by a simple blood test which informs with respect to guanylin and/or uroguanylin levels in the intestine. In some embodiments, levels of proguanylin and/or prouroguanylin, the guanylin and uroguanylin precursors, respectively, that circulate in the blood can be determined and compared to the normal range of levels of proguanylin and/or prouroguanylin, i.e. the range of the amount of levels of proguanylin and/or prouroguanylin which is typically found in healthy, non-obese populations. If the level of levels of proguanylin and/or prouroguanylin is determined to be below either the median or lower limit of the range, an individual can be administered guanylin and/or uroguanylin or proguanylin and/or prouroguanylin can be administered to offset deficiencies of hormone produced by the individual. In some embodiments, levels of proguanylin and/or prouroguanylin in blood samples may be determined using antibody assays such as ELISA assays adapted to provided quatitative results. In some embodiments, levels of proguanylin and/or prouroguanylin in blood samples may be determined using blood samples obtained from an individual 5 minutes to 6 hours following ingestion of fat. In some embodiments, levels of proguanylin and/or prouroguanylin in blood samples may be determined using blood samples obtained from an individual 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, minutes, 170 minutes, 175 minutes, 180 minutes, 185 minutes, 190 minutes, 195 minutes, 200 minutes, 205 minutes, 210 minutes, 215 minutes, 220 minutes, 225 minutes, 230 minutes, 235 minutes, 240 minutes, 245 minutes, 250 minutes, 255 minutes, 260 minutes, 265 minutes, 270 minutes, 275 minutes, 280 minutes, 285 minutes, 290 minutes, 295 minutes, 300 minutes, 305 minutes, 310 minutes, 315 minutes, 320 minutes, 325 minutes, 330 minutes, 335 minutes, 340 minutes, 345 minutes, 350 minutes, 355 minutes, or 360 minutes following ingestion of fat. n some embodiments, levels of proguanylin and/or prouroguanylin in blood samples may be determined using blood samples obtained a period of time within a range selected from the group of every range that can be contain any two of the above listed 5 minute intervals, i.e. 5-10 minutes, 5-15 minutes, etc.

SEQUENCE LISTING

SEQ ID NO: 1: ACAACACATTTTACTGCTGTGAACTTTGTTGTAATCCTGCCTGTGCTGGATGTTAT
encodes AsnAsnThrPheTyrCysCysGluLeuCysCysAsnProAlaCysAlaGlyCysTyr
SEQ ID NO: 2: AsnAsnThrPheTyrCysCysGluLeuCysCysAsnProAlaCysAlaGlyCysTyr
SEQ ID NO: 3: AsnThrPheTyrCysCysGluLeuCysCysTyrProAlaCysAlaGlyCysAsn
SEQ ID NO: 4: ATAGTAGCAATTACTGCTGTGAATTGTGTTGTAATCCTGCTTGTAACGGGTGCTAT
encodes AsnSerSerAsnTyrCysCysGluLeuCysCysAsnProAlaCysAsnGlyCysTyr
SEQ ID NO: 5: AsnSerSerAsnTyrCysCysGluLeuCysCysAsnProAlaCysAsnGlyCysTyr
SEQ ID NO: 6: ProAsnThrCysGluIleCysAlaTyrAlaAlaCysThrGlyCys
SEQ ID NO: 7: AsnAsnThrPheTyrCysCysGluLeuCysCysAsnProAlaCysAlaGlyCys
SEQ ID NO: 8: AsnThrPheTyrCysCysGluLeuCysCysAsnProAlaCysAlaGlyCys
SEQ ID NO: 9: ThrPheTyrCysCysGluLeuCysCysAsnProAlaCysAlaGlyCys
SEQ ID NO: 10: PheTyrCysCysGluLeuCysCysAsnProAlaCysAlaGlyCys
SEQ ID NO: 11: TyrCysCysGluLeuCysCysAsnProAlaCysAlaGlyCys
SEQ ID NO: 12: CysCysGluLeuCysCysAsnProAlaCysAlaGlyCys
SEQ ID NO: 13: AsnThrPheTyrCysCysGluLeuCysCysAsnProAlaCysAlaGlyCysTyr (
SEQ ID NO: 14: ThrPheTyrCysCysGluLeuCysCysAsnProAlaCysAlaGlyCys Tyr
SEQ ID NO: 15: PheTyrCysCysGluLeuCysCysAsnProAlaCysAlaGlyCysTyr
SEQ ID NO: 16: TyrCysCysGluLeuCysCysAsnProAlaCysAlaGlyCysTyr
SEQ ID NO: 17: CysCysGluLeuCysCysAsnProAlaCysAlaGlyCysTyr
SEQ ID NO: 18: AsnThrPheTyrCysCysGluLeuCysCysTyrProAlaCysAlaGly Cys
SEQ ID NO: 19: ThrPheTyrCysCysGluLeuCysCysTyrProAlaCysAlaGlyCys
SEQ ID NO: 20: PheTyrCysCysGluLeuCysCysTyrProAlaCysAlaGlyCys
SEQ ID NO: 21: TyrCysCysGluLeuCysCysTyrProAlaCysAlaGlyCys
SEQ ID NO: 22: CysCysGluLeuCysCysTyrProAlaCysAlaGlyCys
SEQ ID NO: 23: ThrPheTyrCysCysGluLeuCysCysTyrProAlaCysAlaGlyCysAsn
SEQ ID NO: 24: PheTyrCysCysGluLeuCysCysTyrProAlaCysAlaGlyCysAsn
SEQ ID NO: 25: TyrCysCysGluLeuCysCysTyrProAlaCysAlaGlyCysAsn
SEQ ID NO: 26: CysCysGluLeuCysCysTyrProAlaCysAlaGlyCysAsn
SEQ ID NO: 27: AsnSerSerAsnTyrCysCysGluLeuCysCysAsnProAlaCysThrGlyCys
SEQ ID NO: 28: SerSerAsnTyrCysCysGluLeuCysCysAsnProAlaCysThrGlyCys
SEQ ID NO: 29: SerAsnTyrCysCysGluLeuCysCysAsnProAlaCysThrGlyCys
SEQ ID NO: 30: AsnTyrCysCysGluLeuCysCysAsnProAlaCysThrGlyCys
SEQ ID NO: 31: TyrCysCysGluLeuCysCysAsnProAlaCysThrGlyCys
SEQ ID NO: 32: CysCysGluLeuCysCysAsnProAlaCysThrGlyCys
SEQ ID NO: 33: SerSerAsnTyrCysCysGluLeuCysCysAsnProAlaCysThrGlyCysTyr
SEQ ID NO: 34: SerAsnTyrCysCysGluLeuCysCysAsnProAlaCysThrGlyCysTyr
SEQ ID NO: 35: AsnTyrCysCysGluLeuCysCysAsnProAlaCysThrGlyCysTyr
SEQ ID NO: 36: TyrCysCysGluLeuCysCysAsnProAlaCysThrGlyCysTyr
SEQ ID NO: 37: CysCysGluLeuCysCysAsnProAlaCysThrGlyCysTyr
SEQ ID NO: 38: AsnThrPheTyrCysCysGluLeuCysCysAsnProAlaCysAlaGlyCysTyr
SEQ ID NO: 39: AsnThrPheTyrCysCysGluLeuCysCysAlaProAlaCysAlaGlyCysTyr
SEQ ID NO: 40: AsnThrPheTyrCysCysGluLeuCysCysAsnAlaAlaCysAlaGlyCysTyr
SEQ ID NO: 41: AsnThrPheTyrCysCysGluLeuCysCysAsnProAlaCysAlaGlyCys
SEQ ID NO: 42: TyrCysCysGluLeuCysCysAsnProAlaCysAlaGlyCysTyr
SEQ ID NO: 43: TyrCysCysGluLeuCysCysAsnProAlaCysAlaGlyCys
SEQ ID NO: 44: CysCysGluLeuCysCysAsnProAlaCysAlaGlyCysTyr
SEQ ID NO: 45: CysCysGluLeuCysCysAsnProAlaCysAlaGlyCys
SEQ ID NO: 46: GlnAlaCysAspProProSerProProAlaGluValCysCysAspVal
CysCysAsnProAlaCysAlaGlyCys
SEQ ID NO: 47: IleAspCysCysIleCysCysAsnProAlaCysPheGlyCysLeuAsn
SEQ ID NO: 48: SerSerAspTrpAspCysCysAspValCysCysAsnProAlaCysAlaGlyCys
SEQ ID NO: 49: AsnSerSerAsnTyrCysCysGluLeuCysCysTyrProAlaCysThrGlyCysTyr
SEQ ID NO: 50: CysCysAspValCysCysAsnProAlaCysThrGlyCys
SEQ ID NO: 51: CysCysAspValCysCysTyrProAlaCysThrGlyCysTyr
SEQ ID NO: 52: CysCysAspLeuCysCysAsnProAlaCysAlaGlyCysTyr
SEQ ID NO: 53: CysCysGlnLeuCysCysAsnProAlaCysThrGlyCysTyr
SEQ ID NO: 54: ProGlyThrCysGluIleCysAlaTyrAlaAlaCysThrGlyCys
SEQ ID NO: 55: GlnGluAspCysGluLeuCysIleAsnValAlaCysThrGlyCys
SEQ ID NO: 56: AsnAspAspCysGluLeuCysValAsnValAlaCysThrGlyCysLeu
SEQ ID NO: 57: VTVQDGNF SFSLESVKKL KDLQEPQEPR VGKLRNFAPI PGEPVVPILC
SNPNFPEELK PLCKEPNAQE ILQRLEEIAE DPGTCEICAYAACTGC
SEQ ID NO: 58: VYIQYQGF RVQLESMKKL SDLEAQWAPS PRLQAQSLLP
AVCHHPALPQ DLQPVCASQE ASSIFKTLRT IANDDCELCV NVACTGCL

1-6. (canceled)

7. An isolated bacterium comprising a nucleic acid molecule encoding a GCC agonist operably linked to inducible regulatory elements, wherein
if said bacterium is E. coli and said GCC agonist is SEQ ID NO:2. 3 or 5, then said regulatory elements are inducible,
if said bacterium is Y. enterocolitica and said GCC agonist is SEQ ID NO:46 or 48, then said regulatory elements are inducible,
and
if said bacterium is V. cholerae and said GCC agonist is SEQ ID NO 47, then said regulatory elements are inducible.

8. An isolated bacterium of claim 7 wherein said regulatory sequences comprise an inducible promoter.

9. An isolated bacterium of claim 7 wherein said bacterium is auxotrophic in a human large intestine.

10. An isolated culture of bacteria comprising the isolated bacterium of claim 1.

11. A composition comprising a guanylyl cyclase C agonist formulated for large intestine specific release when administered orally to an individual.

12. The composition of claim 11 wherein the guanylyl cyclase C agonist formulated for large intestine specific release when administered orally to an individual by coating it or encapsulating it within a coating.

13. A method of preventing colonic polyps in an individual who has been identified as being at high risk for colonic polyps comprising the step of administering to said individual an effective amount of a composition of claim 11 a to prevent colon polyps.

14. (canceled)

15. A method of treating an individual who has been identified as having colonic polyps comprising the step of administering to said individual, an effective amount of a composition of claim 11 to treat colon polyps.

16. (canceled)

17. A method of preventing colorectal cancer in an individual who has been identified as being at high risk for colorectal cancer comprising the step of administering to said individual an effective amount of a composition of claim 11 to prevent colorectal cancer.

18. (canceled)

19. A method of treating an individual who has been diagnosed as having colorectal cancer comprising the step of administering to said individual an effective amount of a composition of claim 11 to treat colorectal cancer.

20-26. (canceled)

27. The composition of claim 11 wherein said guanylyl cyclase C agonist is a peptide.

28. The composition of claim 11 wherein said guanylyl cyclase C agonist is a peptide selected from the group consisting of: SEQ ID NO:2, 4-57 or 58.

29. The composition of claim 12 wherein said guanylyl cyclase C agonist is a peptide.

30. The composition of claim 12 wherein said guanylyl cyclase C agonist is a peptide selected from the group consisting of: SEQ ID NO:2, 4-57 or 58.


SEQUENCE LISTING (58 sequences)

SEQ ID NO: 1 - DNA, 56 bp, Escherichia coli
acaacacattttactgctgtgaactttgttgtaatcctgcctgtgctggatgttat

SEQ ID NO: 2 - protein, 19 aa, Escherichia coli
NNTFYCCELCCNPACAGCY

SEQ ID NO: 3 - protein, 18 aa, Escherichia coli
NTFYCCELCCYPACAGCN

SEQ ID NO: 4 - DNA, 56 bp, Escherichia coli
atagtagcaattactgctgtgaattgtgttgtaatcctgcttgtaacgggtgctat

SEQ ID NO: 5 - protein, 19 aa, Escherichia coli
NSSNYCCELCCNPACNGCY

SEQ ID NO: 6 - protein, 15 aa, Rattus norvegicus
PNTCEICAYAACTGC

SEQ ID NO: 7 - protein, 18 aa, Artificial Sequence
  peptide fragment 1
NNTFYCCELCCNPACAGC

SEQ ID NO: 8 - protein, 17 aa, Artificial sequence
  peptide  fragment 2
NTFYCCELCCNPACAGC

SEQ ID NO: 9 - protein, 16 aa, Artificial Sequence
  peptide  fragment 3
TFYCCELCCNPACAGC

SEQ ID NO: 10 - protein, 15 aa, Artificial sequence
  peptide fragment 4
FYCCELCCNPACAGC

SEQ ID NO: 11 - protein, 14 aa, Artificial sequence
  peptide  fragment 5
YCCELCCNPACAGC

SEQ ID NO: 12 - protein, 13 aa, Artificial sequence
  peptide fragment 6
CCELCCNPACAGC

SEQ ID NO: 13 - protein, 18 aa, Artificial sequence
  peptide fragment 7
NTFYCCELCCNPACAGCY

SEQ ID NO: 14 - protein, 17 aa, Artificial sequence
  peptide fragment 8
TFYCCELCCNPACAGCY

SEQ ID NO: 15 - protein, 16 aa, Artificial sequence
  peptide fragment 9
FYCCELCCNPACAGCY

SEQ ID NO: 16 - protein, 15 aa, Artificial sequence
  peptide fragment 10
YCCELCCNPACAGCY

SEQ ID NO: 17 - protein, 14 aa, Artificial sequence
  peptide fragment 11
CCELCCNPACAGCY

SEQ ID NO: 18 - protein, 17 aa, Artificial sequence
  peptide fragment 12
NTFYCCELCCYPACAGC

SEQ ID NO: 19 - protein, 16 aa, Artificial sequence
  peptide fragment 13
TFYCCELCCYPACAGC

SEQ ID NO: 20 - protein, 15 aa, Artificial sequence
  peptide fragment 14
FYCCELCCYPACAGC

SEQ ID NO: 21 - protein, 14 aa, Artificial sequence
  peptide fragment 15
YCCELCCYPACAGC

SEQ ID NO: 22 - protein, 13 aa, Artificial sequence
  peptide fragment 16
CCELCCYPACAGC

SEQ ID NO: 23 - protein, 17 aa, Artificial sequence
  peptide fragment 17
TFYCCELCCYPACAGCN

SEQ ID NO: 24 - protein, 16 aa, Artificial sequence
  peptide fragment 18
FYCCELCCYPACAGCN

SEQ ID NO: 25 - protein, 15 aa, Artificial sequence
  peptide fragment 19
YCCELCCYPACAGCN

SEQ ID NO: 26 - protein, 14 aa, Artificial sequence
  peptide fragment 20
CCELCCYPACAGCN

SEQ ID NO: 27 - protein, 18 aa, Artificial sequence
  peptide fragment 21
NSSNYCCELCCNPACTGC

SEQ ID NO: 28 - protein, 17 aa, Artificial sequence
  peptide fragment 22
SSNYCCELCCNPACTGC

SEQ ID NO: 29 - protein, 16 aa, Artificial sequence
  peptide fragment 23
SNYCCELCCNPACTGC

SEQ ID NO: 30 - protein, 15 aa, Artificial sequence
  peptide fragment 24
NYCCELCCNPACTGC

SEQ ID NO: 31 - protein, 14 aa, Artificial sequence
  peptide fragment 25
YCCELCCNPACTGC

SEQ ID NO: 32 - protein, 13 aa, Artificial sequence
  peptide fragment 26
CCELCCNPACTGC

SEQ ID NO: 33 - protein, 18 aa, Artificial sequence
  peptide fragment 27
SSNYCCELCCNPACTGCY

SEQ ID NO: 34 - protein, 17 aa, Artificial sequence
  peptide fragment 28
SNYCCELCCNPACTGCY

SEQ ID NO: 35 - protein, 16 aa, Artificial sequence
  peptide fragment 29
NYCCELCCNPACTGCY

SEQ ID NO: 36 - protein, 15 aa, Artificial sequence
  peptide fragment 30
YCCELCCNPACTGCY

SEQ ID NO: 37 - protein, 14 aa, Artificial sequence
  peptide fragment 31
CCELCCNPACTGCY

SEQ ID NO: 38 - protein, 18 aa, Artificial sequence
  peptide fragment 32
NTFYCCELCCNPACAGCY

SEQ ID NO: 39 - protein, 18 aa, Artificial sequence
  peptide fragment 33
NTFYCCELCCAPACAGCY

SEQ ID NO: 40 - protein, 18 aa, Artificial sequence
  peptide fragment 34
NTFYCCELCCNAACAGCY

SEQ ID NO: 41 - protein, 17 aa, Artificial sequence
  peptide fragment 35
NTFYCCELCCNPACAGC

SEQ ID NO: 42 - protein, 15 aa, Artificial sequence
  peptide fragment 36
YCCELCCNPACAGCY

SEQ ID NO: 43 - protein, 14 aa, Artificial sequence
  peptide fragment 37
YCCELCCNPACAGC

SEQ ID NO: 44 - protein, 14 aa, Artificial sequence
  peptide fragment 38
CCELCCNPACAGCY

SEQ ID NO: 45 - protein, 13 aa, Artificial sequence
  peptide fragment 39
CCELCCNPACAGC

SEQ ID NO: 46 - protein, 25 aa, Yersinia enterocolitica
QACDPPSPPAEVCCDVCCNPACAGC

SEQ ID NO: 47 - protein, 16 aa, Vibrio cholerae
IDCCICCNPACFGCLN

SEQ ID NO: 48 - protein, 18 aa, Yersinia enterocolitica
SSDWDCCDVCCNPACAGC

SEQ ID NO: 49 - protein, 19 aa, Artificial sequence
  peptide fragment 40
NSSNYCCELCCYPACTGCY

SEQ ID NO: 50 - protein, 13 aa, Artificial sequence
  peptide fragment 41
CCDVCCNPACTGC

SEQ ID NO: 51 - protein, 14 aa, Artificial sequence
  peptide fragment 42
CCDVCCYPACTGCY

SEQ ID NO: 52 - protein, 14 aa, Artificial sequence
  peptide fragment 43
CCDLCCNPACAGCY

SEQ ID NO: 53 - protein, 14 aa, Artificial sequence
  peptide fragment 44
CCQLCCNPACTGCY

SEQ ID NO: 54 - protein, 15 aa, Homo sapiens
PGTCEICAYAACTGC

SEQ ID NO: 55 - protein, 15 aa, Didelphis virginiana
QEDCELCINVACTGC

SEQ ID NO: 56 - protein, 16 aa, Artificial sequence
  peptide fragment 45
NDDCELCVNVACTGCL

SEQ ID NO: 57 - protein, 94 aa, Homo sapiens
VTVQDGNFSFSLESVKKLKDLQEPQEPRVGKLRNFAPIPGEPVVPILCSNPNFPEELKPLCKEPNAQEILQRLEEIAEDPGTCEICAYAACTGC

SEQ ID NO: 58 - protein, 86 aa, Homo sapiens
VYIQYQGFRVQLESMKKLSDLEAQWAPSPRLQAQSLLPAVCHHPALPQDLQPVCASQEASSIFKTLRTIANDDCELCVNVACTGCL